(19) United States
(12) Patent Application Publication
(10) Pub. No.: US 2026/0060825 A1
(43) Pub. Date: Mar. 5, 2026

(54) STENTS

(71) Applicant: OTSUKA MEDICAL DEVICES CO., LTD., Tokyo (JP)

(72) Inventors: Kevin Heraty, Galway (IE); Hugh Mc Sweeney, Galway (IE); Coroline Higgins, Galway (IE)

(73) Assignee: Otsuka Medical Devices Co., Ltd., Tokyo (JP)

(21) Appl. No.: 19/120,900

(22) PCT Filed: Oct. 2, 2023
(86) PCT No.: PCT/IB2023/059869

§ 371 Date: Apr. 14, 2025

(30) Foreign Application Priority Data
Oct. 14, 2022 (GB) 2215239.1

(51) Int. Cl. A61F 2/915

(52) U.S. Cl. CPC A61F 2/915; A61F 2002/91558; A61F 2002/91566; A61F 2002/91583; A61F 2220/0008; A61F 2220/0025; A61F 2230/0006; A61F 2230/0069; A61F 2240/001; A61F 2250/0039

(57) ABSTRACT

A stent comprises a longitudinal series of circumferential tubular segments that are separated by, and alternate with, gaps along the length of the stent between opposed end segments of the series. Each segment of the series comprises struts that are disposed in a circumferentially extending waveform arrangement. Successive segments of the series are interconnected by connectors that bridge the respective gaps. Segments of the series disposed between the end segments comprise at least one anchor segment that is pre-formed with a radially enlarged configuration relative to at least one interconnected adjacent body segment of lesser radius. In use, the anchor segments engage a surrounding vessel wall to resist migration of the stent, assisted by serrations defined by the struts of the waveform arrangement. An anchor segment is connected to a body segment by connectors extending between facing troughs of the waveform arrangements of those successive segments.

This invention relates to stents, especially stents for the treatment of occlusions in blood vessels. The invention relates particularly to stents that have a skeletal frame structure derived from a pattern of slits that is cut or otherwise formed in a tube before the tube is expanded to open out the structure.

Stents are typically inserted into a vessel in a narrowed initial state and are then expanded radially to support the surrounding wall of the vessel so as to restore or maintain its patency. Some stents self-expand elastically when released from within a sleeve or catheter. Others expand plastically either when activated, for example by heat acting on a shape-memory alloy, or by being pressed radially outwardly from within using an instrument such as a balloon catheter.

The genesis of the invention relates to the treatment of blocked iliac and femoral veins using stents to establish patency and adequate blood flow, and the need to prevent migration of such stents along the veins in which they are placed. The invention has special benefits in that context and more generally in the context of venous applications. However, the inventive concept embraces other stent applications in which longitudinal migration could be a problem, including arterial applications.

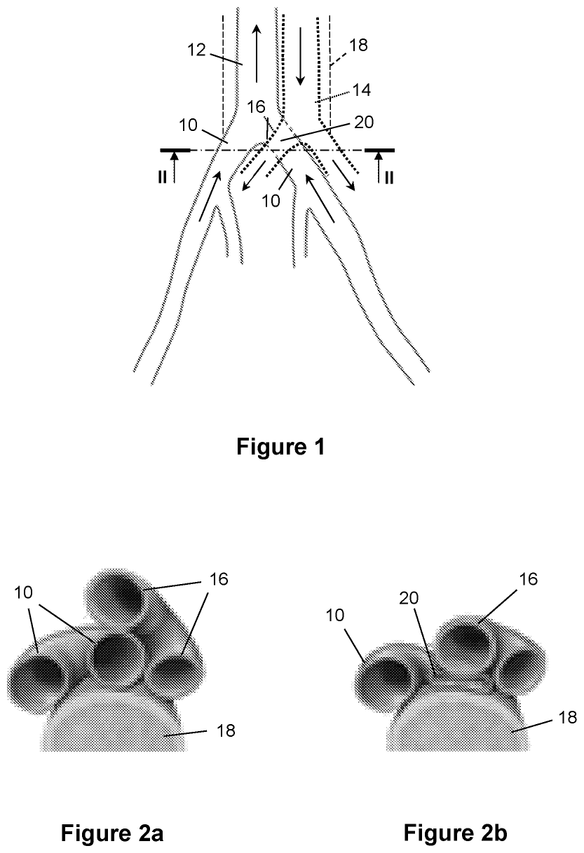

FIG. 1 shows, in solid lines, the convergence of the common iliac veins 10 into the lower end of the inferior vena cava 12 in a human individual. The iliac veins 10 drain blood from the lower abdomen and the lower limbs into the inferior vena cava 12 and from there to the heart. The inferior vena cava 12 extends up to the heart beside the abdominal aorta 14, shown here in dotted lines, which reciprocally carries blood from the heart to the lower abdomen and the lower limbs. The abdominal aorta 14 bifurcates at its lower end into the common iliac arteries 16.

The inferior vena cava 12 and the abdominal aorta 14 lie beside each other in front of the vertebral column 18, whose outline is represented schematically in FIG. 1 by parallel dashed lines. The bifurcation of the abdominal aorta 14 is typically aligned with the fourth vertebra of the lumbar spine, known as L4. As FIG. 1 is a front view, the abdominal aorta 14, shown here to the right, lies to the left of the inferior vena cava 12 from the individual's perspective. Similarly, the individual's left common iliac vein 10 is shown to the right and so on.

It will be apparent from FIG. 1 that the right common iliac artery 16 crosses and overlies the left common iliac vein 10. The positional relationship between the common iliac veins 10 and the common iliac arteries 16 can also be appreciated in the cross-sectional views of FIGS. 2a and 2b.

FIGS. 2a and 2b show that the common iliac veins 10 and the common iliac arteries 16 also lie in front of the vertebral column 18, typically in front of the lowermost lumbar vertebra, known as L5. It follows that the left common iliac vein 10 is disposed between the vertebral column 18 and the overlying right common iliac artery 16, where the right common iliac artery 16 crosses over the left common iliac vein 10. The left common iliac vein 10 is therefore susceptible to compression by the right common iliac artery 16 and hence to a substantial reduction of patency in this region, as shown in FIG. 2b in contrast to its uncompressed state in FIG. 2a.

Luminal compression of the left common iliac vein is common in the population, occurring in nearly a quarter of healthy individuals. The condition is largely asymptomatic but, in some cases, the loss of patency becomes clinically significant and gives rise to May-Thurner syndrome, also known as iliac vein compression syndrome. May-Thurner syndrome is characterised by swelling, pain, deep venous thrombosis, skin discolouration, ulcers and/or the formation of collateral blood vessels, all caused by changes in venous flow or venous pressure arising from limited blood flow out of the affected leg. Its symptoms can severely impact an individual's quality of life.

When properly diagnosed, luminal compression of the common iliac vein is referred to as a non-thrombotic iliac vein lesion or NIVL 20, which typically occurs at or just upstream of the junction between the left common iliac vein 10 and the inferior vena cava 12 as shown in FIG. 1.

Venous stenting has become the standard technique for treating occlusive blockages in the iliac and femoral veins. In particular, patency of the common iliac vein can be restored successfully by placing a stent device across the blockage of a NIVL. The stent pushes the wall of the vein back against the overlying artery to restore normal blood flow, thereby enabling venous drainage.

Various stents currently on the market have been used to treat NIVLs. Braided stents have been used but are largely ineffective for this purpose as they have inadequate radial strength or crush resistance. Consequently, straight tubular stents of laser-cut nitinol are preferred to treat NIVLs. Such stents are cut in various patterns to create skeletal frame structures, when expanded or opened out, for which various functional advantages have been claimed.

On occasion, venous stents can become dislodged and then migrate downstream through the vasculature. A migrated stent may require another surgical or endoscopic procedure to retrieve it, which increases risk and inconvenience to the patient.

Migration can be particularly serious when a stent is used to treat a NIVL because a dislodged stent is apt to migrate into the inferior vena cava and from there to the heart, where it could threaten the patient's life. In this respect, the cross-sectional luminal area of the inferior vena cava increases in the downstream direction from the common iliac veins toward the heart, hence presenting little resistance to continued migration of a dislodged stent away from a NIVL.

Stent migration has become a particular problem for laser cut stents placed in NIVL patients. There have been numerous occurrences of venous stent migration in recent years. In some such cases, stents have been noted to migrate within a few hours of the procedure and in other cases, up to six months after the procedure.

In this respect, a stent is most susceptible to becoming dislodged if it is undersized relative to the lumen or internal diameter of the vessel in which it is placed. Thus, before a stent is placed across a venous lesion such as a NIVL, the vein diameter must be measured carefully to ensure that a stent of the appropriate diameter is chosen. However, vessel sizing is a difficult and imprecise art. Veins are challenging to measure accurately, especially when deformed into luminal compression, and the vein diameter can change due to various factors such as the patient's level of hydration, their body position (supine vs standing), and a breathing manoeuvre or the Valsalva manoeuvre performed at the time of measurement. As a result, there is a risk that an undersized stent can be placed in a vein and, if it migrates, eventually become lodged in the heart.

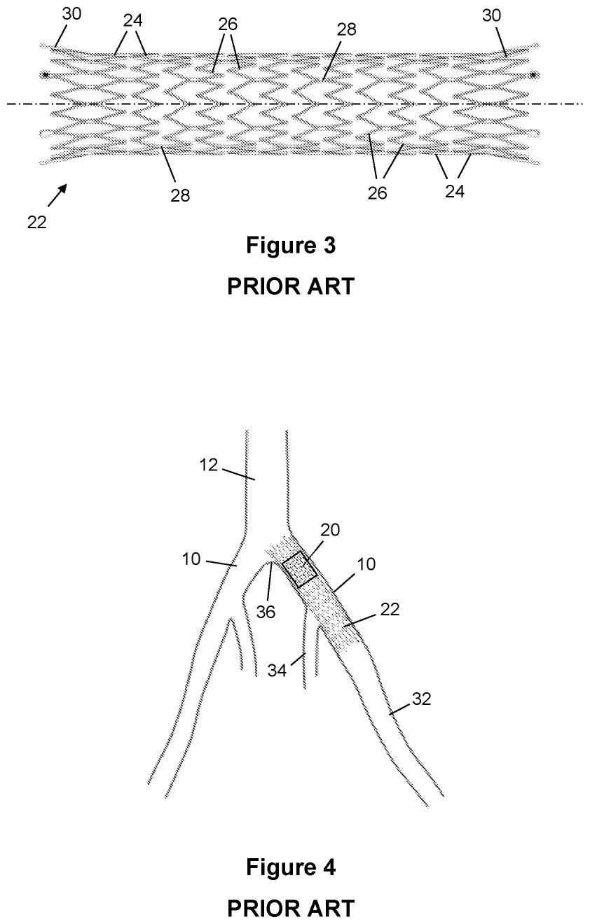

FIG. 3 shows an example of the ‘Venovo’ stent offered by BD (Becton, Dickinson and Company). Those trade marks are acknowledged. The Venovo device is a laser-cut nitinol stent 22 that comprises a series of circumferential crowns or segments 24 each comprising a zig-zag array of struts 26 defining peaks and troughs. The successive segments 24 are joined by short longitudinal connectors 28 extending from peaks of one segment 24 to peaks of the next segment 24. Unlike the substantially uniform cross-sectional diameter of other such stents on the market, the end portions 30 of the stent 22 are flared outwardly. Thus, the end portions 30 defined by the terminal segments 24 of the stent 22 are generally frusto-conical, increasing in diameter outwardly in opposite longitudinal directions.

Whilst the flared end portions 30 of the Venovo stent 22 are intended to reduce the risk of migration, migrations of that stent type have still been observed in the NIVL patient population. To understand why this is so, reference is made to FIG. 4 which shows a Venovo stent 22 in situ. There, the stent 22 is positioned to treat a NIVL 20 positioned at or close to the junction between the common iliac vein 10 and the inferior vena cava 12.

The proximal end of the stent 22 is at or slightly downstream of the junction between the common iliac vein 10 and the inferior vena cava 12, whereas the distal end of the stent 22 is at an upstream location wholly within the iliac vein 10. Indeed, in this example, the distal end of the stent is in the external iliac vein 32 upstream of its junction with the internal iliac vein 34.

A flared end portion 30 will work best to resist movement only in the direction of its outward flare. In this respect, the proximal end of the stent 22 is the leading end relative to the direction of blood flow. The flared proximal end portion 30 will engage the vessel wall and will tend to widen or splay and therefore dig into or engage mechanically with the vessel wall more firmly with continued longitudinal movement in the downstream or proximal direction.

Conversely, the trailing distal end portion 30 of the stent 22 lacks a similar advantage because it tapers rather than widens in the direction of movement. Consequently, the trailing end of the stent will tend to narrow and hence to disengage from the vessel wall with continued longitudinal movement, doing little to resist that movement. In that situation, only minimal friction can arise from radially outward force exerted by the distal rim of the trailing end against the vessel wall.

It follows that location of the stent 22 against migration relies principally on the flared proximal end portion 30 of the stent 22. As noted above, the proximal end portion 30 is located at the downstream or leading end of the stent 22 with respect to the blood flow and therefore at or close to the ridge or carina 36 of the bifurcation between the inferior vena cava 12 and the left and right common iliac veins 10.

A flared end portion 30 will work best to restrict longitudinal movement of a stent 22 when contacting the surrounding wall of a vessel whose lumen is circular or elliptical in cross-section. In that scenario, the rim at the extremity of the flared end portion 30 can make consistently firm contact with the vessel wall around the full circumference of the vessel. However, in the region of the bifurcation of the inferior vena cava 12, the cross-section of the vessel is non-circular and its cross-sectional area increases moving proximally or downstream. Indeed, the geometry of the vessel wall changes constantly and in multiple directions, particularly around the carina 36 disposed between the left and right common iliac veins 10. Around the carina 36, especially, the proximal rim at the leading end of the flared end portion 30 may not make optimal contact around its full circumference with the vessel wall. In that case, the flared end portion 30 at the leading or proximal end of the stent 22 may offer inadequate resistance to axial movement in the downstream direction.

US 2007/0142907 describes a prosthetic implant for treating a diseased aortic valve. The implant comprises rings that are adjustable to suit valves of different sizes. The diameters of the adjustable rings are enlarged postoperatively by the surgeon placing the implant. Consequently, enlargement of the rings depends on the skill of the surgeon. Furthermore, the adjustable rings described remain susceptible to unintended variation in diameter when in situ, for example, due to magnetism during an MRI procedure.

WO 2018/110999 discloses a pancreatic stent comprising a body and a bending portion. During implantation, the stent is axially compressed causing the bending portion to flare radially to the point where a flange feature is created extending about the full circumference of the stent body. Such a stent is not suitable for a venous or arterial application.

It is against this background that the present invention has been devised. In one aspect, the invention resides in a stent comprising a tubular body extending longitudinally between a proximal open end and a distal open end, wherein the tubular body has an enlarged portion extending longitudinally between the proximal open end and the distal open end. The enlarged portion is disposed between longitudinally adjacent portions of the tubular body and has a radially enlarged configuration relative to at least both of the longitudinally adjacent portions. The enlarged portion may be pre-formed with a radially enlarged configuration relative to the proximal open end and the distal open end.

The invention also resides in a stent comprising a longitudinal series of circumferential tubular segments that are separated by, and alternate with, gaps along the length of the stent between opposed end segments of the series. Each segment of the series comprises struts that are disposed in a circumferentially extending waveform arrangement. Successive segments of the series are interconnected by connectors that bridge the respective gaps. Segments of the series disposed between the end segments comprise at least one anchor segment that is pre-formed with a radially enlarged configuration relative to at least one interconnected adjacent body segment of lesser radius.

At least one anchor segment may be of substantially constant diameter along its length in a longitudinal direction or may be flared along its length in a longitudinal direction. In the latter case, the or each flared anchor segment may expand in a longitudinal direction toward one of the end segments, or at least two anchor segments may be flared in opposed longitudinal directions.

The connectors suitably extend between respective apices of the waveform arrangements of successive segments. For example, trough-to-trough connectors may extend between facing troughs of the waveform arrangements of successive segments.

In that case, the trough-to-trough connectors may connect at least one anchor segment to at least one body segment of the series. Conversely, peak-to-peak connectors may extend between facing peaks of the waveform arrangements of successive segments. Such peak-to-peak connectors may connect successive anchor segments and/or successive body segments of the series. The connectors may have opposed bends or inflections in succession along their length.

At least two of the anchor segments may be grouped in longitudinal succession. At least one anchor segment may extend longitudinally to a substantially different extent than at least one other segment of the series.

A stepped outer profile may be defined between at least one anchor segment and an interconnected segment of the series. The stepped outer profile may, for example, comprise a radially-extending circumferential shoulder, and may be circumferentially serrated. Elegantly, serrations of the stepped outer profile may be defined by the waveform arrangement of struts of the anchor segment.

The stent may comprise a group of anchor segments whose radius increases and/or decreases along the group. At least two anchor segments may be spaced apart longitudinally along the series and separated by least one body segment. The or each body segment may be of substantially the same diameter as the end segments.

The inventive concept also embraces a method of manufacturing a stent, that method comprising: inserting a cylindrical mandrel into a lumen of the stent, the mandrel comprising at least one radially-protruding circumferential band; aligning at least one portion of the stent longitudinally with the or each band of the mandrel, the or each of those aligned portions of the stent being inboard of opposed end portions of the stent; and by effecting relative radial movement between the stent and the mandrel, forming the stent around the mandrel such that the or each portion aligned longitudinally with the or each band is enlarged radially relative to other portions that are not aligned longitudinally with the or each band.

For a segmented stent, the manufacturing method may comprise: inserting a cylindrical mandrel into a lumen of the stent, the mandrel comprising at least one radially-protruding circumferential band and the stent comprising a longitudinal series of interconnected circumferential tubular segments; aligning at least one of the segments of the stent longitudinally with the or each band of the mandrel, the or each of those aligned segments being inboard of opposed end segments of the stent; and by effecting relative radial movement between the segments and the mandrel, forming the segments around the mandrel such that the or each segment aligned longitudinally with the or each band is enlarged radially relative to other segments that are not aligned longitudinally with the or each band.

At least one circumferential edge of the or each band may be aligned with a respective gap between successive segments of the series. In that case, connectors extending between the successive segments may be accommodated in respective angularly-spaced grooves of the band intersecting the or each circumferential edge.

Each segment of the series may comprise struts that are disposed in a circumferentially-extending waveform arrangement. In that case, interconnection between a segment aligned with the band and an adjacent segment not aligned with the band may be effected by connectors that extend between facing troughs of their waveform arrangements. Conversely, interconnection between segments of a group of segments all aligned with one band may be effected by connectors that extend between facing peaks of their waveform arrangements.

A stepped profile may be formed between a portion of the stent that is aligned with the, or one of the, bands and an adjoining portion that is not aligned with that band. A flared profile may be formed in the or each portion that is aligned with the or each band. A step may be formed between a portion with a flared profile and an adjacent portion of the stent.

A group of two or more successive segments of the series may be aligned longitudinally with one band that is aligned with all segments of the group. The segments of the group may be pressed into conformity with a concave formation of the band by applying radially inward pressure locally to connectors extending between those segments.

The stent of the invention, for example comprising a longitudinal series of interconnected tubular segments, enables a method of anchoring the stent when the segments are placed together in a patient's body. That method comprises focusing or concentrating outward radial pressure of the stent on a surrounding anatomical structure in which the stent is placed, that pressure being focused via at least one tubular segment or portion of the stent that is pre-formed with a radially enlarged configuration relative to at least one adjoining or interconnected adjacent segment or portion of lesser radius, the radially enlarged segment or portion being longitudinally inboard of end segments or portions of the stent.

The stent may, for example, be placed to treat a non-thrombotic iliac vein lesion. Thus, the surrounding anatomical structure may be an iliac vein, in which case the or each radially enlarged segment or portion of the stent may be aligned with the iliac vein or more specifically with the common iliac vein. A proximal end segment or portion of the stent may protrude from the iliac vein into the inferior vena cava.

More generally, the surrounding anatomical structure may be a blood vessel and at least one radially enlarged segment or portion of the stent may flare in a downstream direction with respect to blood flow in the vessel.

The surrounding anatomical structure may be engaged with an edge of the or each radially enlarged segment or portion, for example with serrations of that edge. The serrations may be defined by struts of the radially enlarged segment or portion that are arranged as a circumferentially extending waveform.

In summary, it is an object of the invention to reduce the risk of migration of a stent device. This is achieved by increasing an aspect of the external diameter of the stent. The invention contemplates various ways to achieve expansion of focal diameter, as will be described below. Radial expansion may, for example, be in the order of 1 mm compared with adjacent portions to either side of an expanded portion.

A local increase of focal diameter in stents of the invention provides one or more additional anchor points for the stent with minimal changes to the manufacturing process of the stent and to the design, operation and manufacture of a suitable stent delivery system. In use, the or each anchor point engages a surrounding vessel wall to resist migration of the stent, assisted by serrations defined by the struts in a waveform arrangement. Not only does this strengthen engagement between the stent and the surrounding vessel wall but also makes the stent more tolerant of incorrect sizing and especially under-sizing. Focal diameter may be increased or may otherwise vary at one or more locations along the length of the stent.

To put the invention into context, reference has already been made to FIGS. 1 to 4 of the accompanying drawings, in which:

FIG. 1 is a schematic diagram showing the convergence of the common iliac veins into the inferior vena cava, superimposed by the bifurcation of the abdominal aorta into the common iliac arteries and superimposed on the lumbar region of the vertebral column;

FIGS. 2a and 2b are cross-sectional views on line II-II of FIG. 1, FIG. 2a showing normal vein anatomy and FIG. 2b showing the left common iliac vein compressed against the vertebral column by the overriding right common iliac artery;

FIG. 3 is a side view of a prior art stent that can be used to restore the patency of the common iliac vein; and

FIG. 4 is a schematic diagram showing the prior art stent in situ within the left common iliac vein.

In order that the invention may be more readily understood, reference will now be made, by way of example, to the remainder of the accompanying drawings in which:

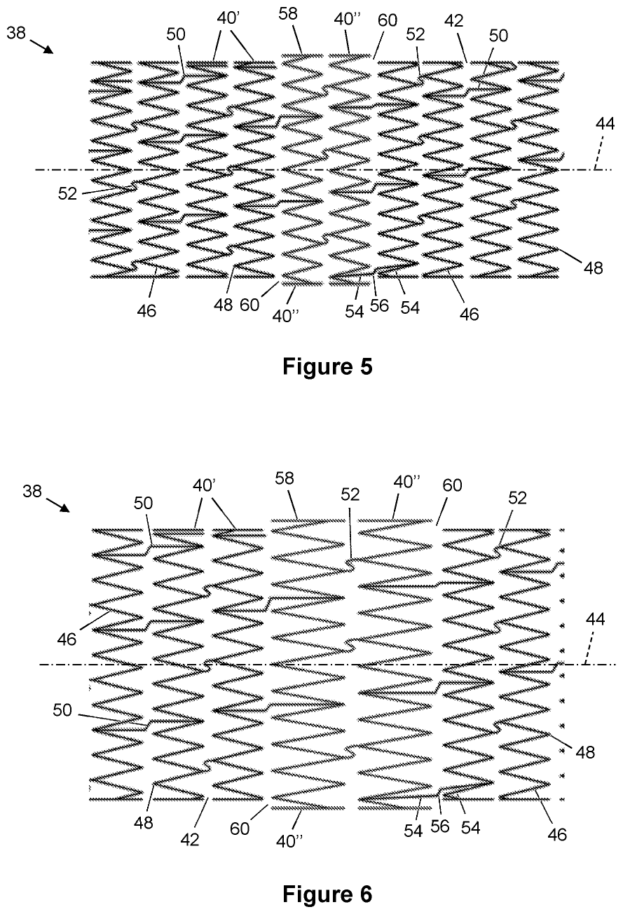

FIGS. 5 and 6 are schematic side views of first and second embodiments of the invention, each showing a portion of a stent;

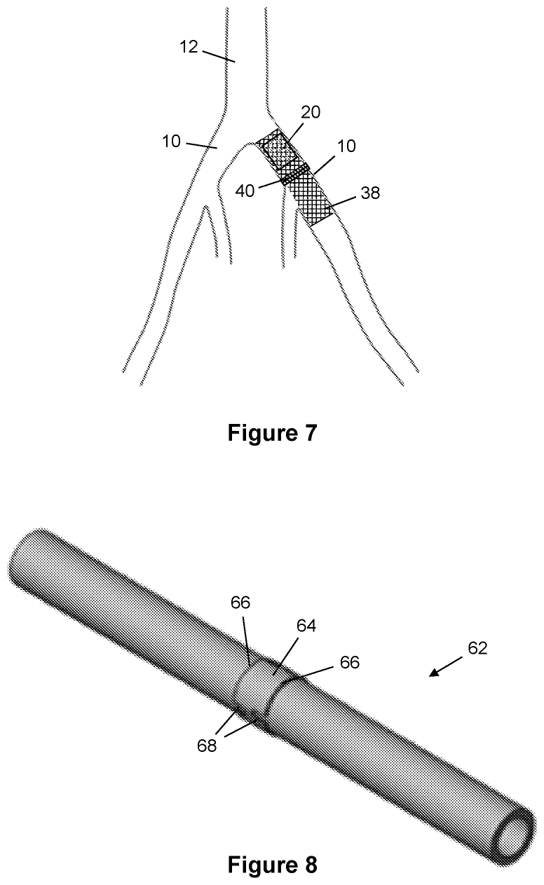

FIG. 7 is a schematic diagram showing a stent of the invention in situ within the left common iliac vein;

FIG. 8 is a perspective view of a mandrel for forming a stent in accordance with the first embodiment of the invention;

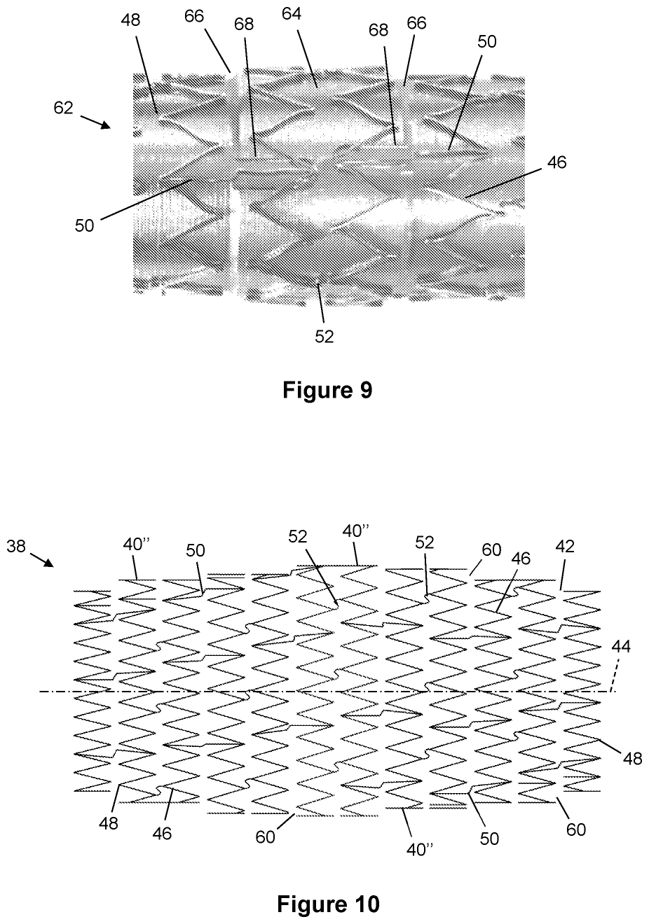

FIG. 9 is an enlarged detail side view of the mandrel of FIG. 8 when forming the stent;

FIG. 10 is a schematic side view of a portion of a stent in a third embodiment of the invention;

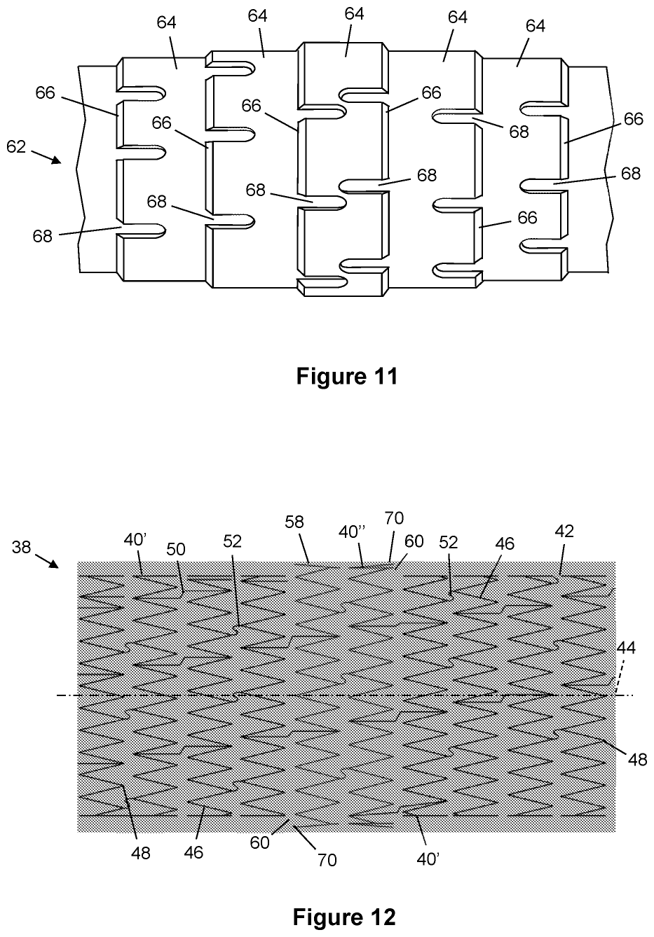

FIG. 11 is a schematic side view of a mandrel for forming the stent illustrated in FIG. 10;

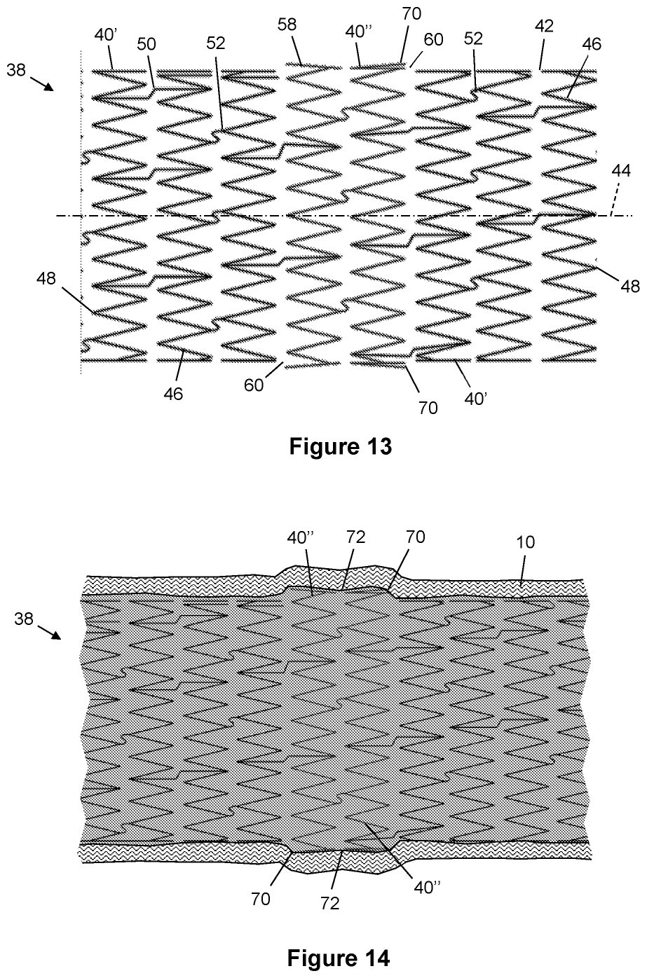

FIGS. 12 and 13 are schematic side views of fourth and fifth embodiments of the invention, each showing a portion of a stent;

FIG. 14 is a schematic side view in longitudinal section showing how the stent illustrated in FIG. 12 engages a surrounding vessel in a patient's body;

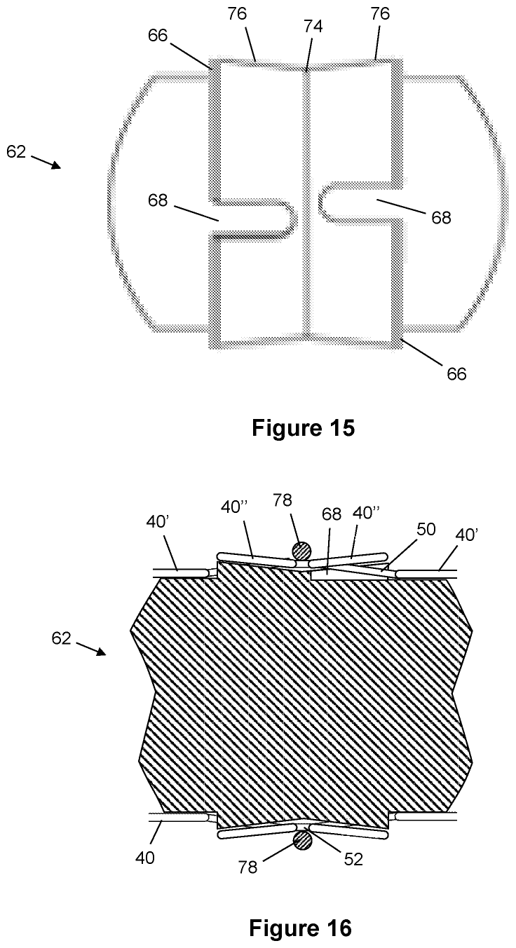

FIG. 15 is an enlarged detail side view of a portion of a mandrel for forming a stent illustrated in FIG. 12;

FIG. 16 corresponds to FIG. 15 but shows the mandrel in longitudinal section and in use when forming the stent illustrated in FIG. 12;

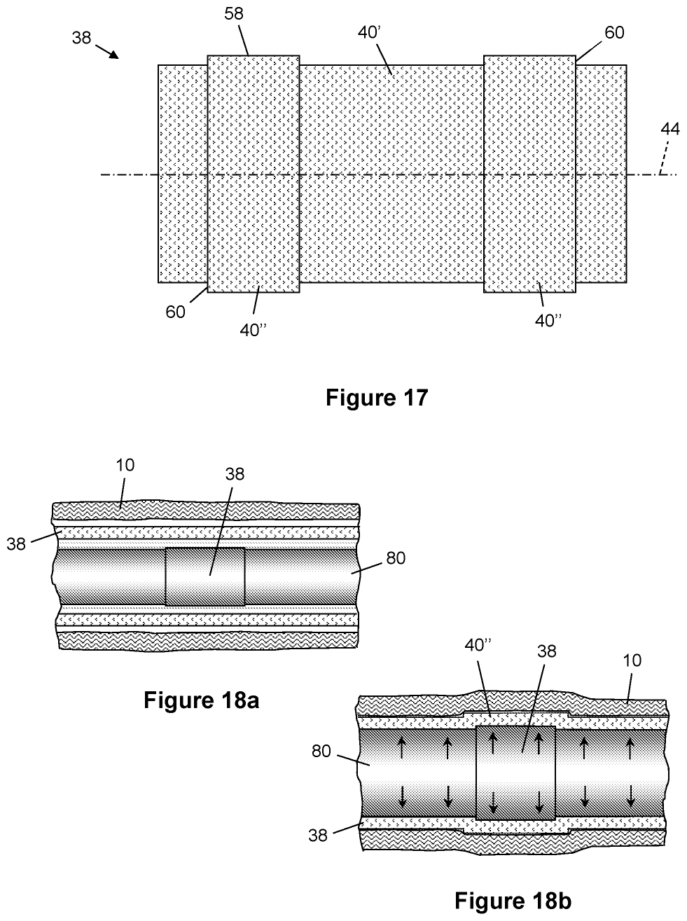

FIG. 17 is a schematic side view of a portion of a stent in a sixth embodiment of the invention; and

FIGS. 18a and 18b are a sequence of schematic side views that show a balloon-expandable stent being formed within a blood vessel by expansion of an internal balloon with a stepped profile.

FIGS. 5, 6, 10, 12 and 13 are schematic drawings that show portions of various embodiments of stents 38. Each stent 38 comprises an open skeletal frame that is formed when an elongated tube is expanded radially into the state shown in these drawings. The tube may, for example, be formed of nitinol laser-cut with a pattern of slits that define members of the frame between the slits when the tube is expanded.

It is emphasised that FIGS. 5, 6, 10, 12 and 13 are schematic drawings to illustrate principles of the invention rather than showing full detail of practical embodiments. For example, in practice, each slit will conveniently define the boundaries of adjacent frame members divided by the slit so that those members have width, shape and complementary or matching outlines defined by the shape of the slit.

The expanded state of the stent 38 as shown in FIGS. 5, 6, 10, 12 and 13 may be an in-use state that is itself sufficient to support a surrounding vessel wall or may be an insertion state that requires further radial expansion into an in-use state when in situ, for example using a balloon catheter.

Each stent 38 shown in FIGS. 5, 6, 10, 12 and 13 comprises a longitudinal array or series of rings, circumferential or tubular segments or crowns 40, distributed along the length of the stent 38 and spaced apart by interstitial circumferential gaps 42. The length of the stent 38 and hence the number of crowns 40 is indeterminate. Only a few of potentially several such crowns 40 are shown in these drawings.

Each crown 40 is circumferentially continuous and, in end view, is rotationally symmetrical about a central longitudinal axis 44 of the stent 38. Each crown 40 also extends parallel to the central longitudinal axis 44 and therefore defines a respective portion of the length of the stent 38, in conjunction with the width of the gaps 42 between neighbouring crowns 40 along that axis 44.

Each crown 40 comprises a circumferentially-extending zig-zag arrangement of struts 46 being the principal members of the skeletal frame. The zig-zag arrangement may also be described as a triangular waveform oscillating circumferentially around the crown 40. In these examples, the rise and fall of the waveform is symmetrical but an asymmetric waveform such as a sawtooth is also possible.

Each strut 46 is inclined relative to a line intersecting the circumference of the associated crown 40 and extending parallel to the central longitudinal axis 44. The inclination of each strut 46 opposes the inclination of the adjoining struts 46 of the same crown 40. In these examples, each strut 46 of a crown 40 is substantially straight and is of substantially the same length as other struts 46 of the same crown 40.

The apices 48 where the struts 46 of each crown 40 join in circumferential succession define peaks and troughs when viewed from the perspective of neighbouring crowns 40. The peaks are apices 48 that are relatively close to a neighbouring crown 40 whereas troughs are apices 48 that are relatively far from a neighbouring crown 40. The peaks and troughs of each crown 40 alternate circumferentially around the crown 40.

As the members of the skeletal frame are formed and defined between slits in a tubular workpiece, it follows that the waveform shape of each crown 40 complements the shape of the, or each, neighbouring crown 40. Thus, before longitudinal expansion of a slit tube to form the stent 38, the successive crowns 40 are in nested relation, with the peaks of each crown 40 aligned with and received by the troughs of a neighbouring crown 40. Consequently, the peaks and troughs of a crown 40 are offset angularly or indexed about the central longitudinal axis 44 relative to the peaks and troughs of the, or each, neighbouring crown 40. In the case of a symmetrical waveform such as that shown in FIGS. 5, 6, 10, 12 and 13, the angular offset from one crown 40 to the next is half a wavelength.

The crowns 40 are joined to the, or each, adjacent crown 40 by sets of connectors 50, 52 that are distributed circumferentially around the central longitudinal axis 44. The connectors 50, 52 extend longitudinally to bridge the gaps 42 between successive crowns 40 and so serve as secondary members of the skeletal frame. More specifically, the stent arrangements shown in FIGS. 5, 6, 10, 12 and 13 comprise two types of connectors 50, 52 between successive crowns 40, namely: long connectors 50 that extend from a trough of one crown 40 to a closest trough of an adjacent crown 40; and short connectors 52 that extend from a peak of one crown 40 to a closest peak of an adjacent crown 40.

The sets of long and short connectors 50, 52 alternate longitudinally, in that one gap 42 is bridged by a set of short connectors 52, the next gap 42 is bridged by a set of long connectors 50 and so on in sequence along the length of the stent 38. The result is that adjacent crowns 40 are joined together as a pair by short connectors 52 and that pair of crowns 40 is joined to one or two adjacent crowns 40 by long connectors 50.

In these examples, the connectors 50, 52 are spaced apart by more than the wavelength of the crown 40. Consequently, not all of the peaks and troughs are joined by connectors 50, 52. Specifically, in these examples, the angular spacing between the connectors 50, 52 alternates circumferentially between every third peak or trough and every fourth peak or trough. Thus, the connectors 50, 52 that bridge a gap 42 are not necessarily spaced equiangularly; in other words, the angular spacing between successive connectors 50, 52 in a gap 42 can vary circumferentially.

It will also be apparent that longitudinally successive sets of the long connectors 50 are offset angularly or indexed circumferentially about the central longitudinal axis 44, as are successive sets of the short connectors 52. The angular offset from one set of connectors 50, 52 to the next set of like connectors 50, 52 is one wavelength of the waveforms defining the crowns 40, hence being a rotation equal to the circumferential distance between successive peaks or troughs.

As the successive crowns 40 are in nested relation before the stent 38 is expanded from a slit tube, the peaks of a crown 40 are substantially aligned in a longitudinal direction with the troughs of a neighbouring crown 40. Consequently, there is an angular offset between the peaks and the troughs of successive crowns 40 that face each other across the gaps 42 between the crowns 40. In these examples, that angular offset is about half a wavelength. It follows that the connectors 50, 52 must allow for the resulting angular offset between their ends at the apices 48 defining the peaks and the troughs.

The connectors 50, 52 allow for the angular offset between their ends either by being inclined relative to the longitudinal direction or, as in these examples, being curved or kinked. Specifically, the short connectors 52 in these examples have an S-shape, being curved continuously through mutually-opposed inflections. Conversely, the long connectors 50 have straight longitudinal portions 54 joined by a central chicane 56 comprising bends in mutually-opposed directions.

These configurations of the connectors 50, 52 have the benefit of conferring flexibility on the stent 38, including longitudinal extensibility to allow the stent 38 to bend along its length and therefore to extend on the extrados or outboard side of the central longitudinal axis 44. By virtue of their greater length making them easier to bend along their length, the long connectors 50 contribute flexibility additional to that provided by the short connectors 52.

Flexibility in the connectors 50, 52 from one crown 40 to the next is advantageous not only in use, so that the stent 38 can more readily conform to internal contours of the vasculature, but also in manufacture so that the stent 38, as manufactured, can be shaped in accordance with the invention without experiencing excessive local stress. The long connectors 50, especially, also effectively decouple radially-enlarged anchor segments or crowns 40″, whether those crowns 40″ are flared or otherwise, from immediately adjacent body segments or crowns 40′of lesser radius. This allows those successive crowns 40 to move and flex independently of each other to a helpful extent during manufacture, placement and in use.

FIGS. 5 and 6 show embodiments of a stepped stent 38 in which a pair of anchor crowns 40″ joined peak-to-peak by short connectors 52 are expanded radially to a greater extent than neighbouring body crowns 40′disposed outboard of that pair to each side. Those body crowns 40′are joined to respective anchor crowns 40″ of the pair by respective long connectors 50 extending trough-to-trough. The flexibility of the long connectors 50 to deflect or deform in response to axial and bending loads accommodates the radial expansion of the inboard pair of anchor crowns 40″ without experiencing excessive stress.

In the embodiments of FIGS. 5 and 6, the radially expanded pair of anchor crowns 40″ remain of substantially uniform diameter along their length. Thus, those anchor crowns 40″ have radially outermost circumferential faces 58 that are straight-sided and parallel to the central longitudinal axis 44 when viewed in longitudinal section.

Nevertheless, by virtue of their radial enlargement, the anchor crowns 40″ protrude radially like a flange from the surrounding cylindrical outline of the stent 38 defined by the outboard body crowns 40′.

The flange-like radial protrusion of the anchor crowns 40″ defines external formations 60 being steps, edges or shoulders between the lesser radius of the body crowns 40′ and the greater radius of the expanded anchor crowns 40″ disposed between them. It will be apparent that these circumferential external formations 60 will improve mechanical and frictional engagement of the stent 38 with a surrounding vessel in use and, in particular, will resist longitudinal movement and hence migration of the stent 38.

The protruding anchor crowns 40″ also focus the radial expansion force of the stent 38 on a smaller portion of the overall outer surface area of the stent 38. This increases the outward pressure acting against the interior of the surrounding vessel wall to enhance mechanical and frictional engagement of the stent 38 with the vessel.

To further benefit, the outboard apices 48 or peaks of the waveforms of the anchor crowns 40″ lend a serrated profile to the external formations 60. When the stent 38 is in situ within a common iliac vein 10 or other vessel as represented in FIG. 7, the serrated external formations 60 of the anchor crowns 40″ dig into and engage mechanically with the surrounding wall of the vein 10. Consequently, those formations 60 have a similar locating function to the flared ends 30 of the prior art stent 22 shown in FIGS. 3 and 4, but the inboard location of the formations 60 has further advantages.

In this respect, it will be apparent that the inboard position of the anchor crowns 40″ and their external formations 60, hence away from the ends of the stent 38, may better engage the surrounding vessel wall than a corresponding enlargement at an end of a stent. In this respect, it will be recalled that the flared proximal end 30 of the prior art stent 22 shown in FIGS. 3 and 4 may not adequately engage the surrounding vessel wall in the region of the carina 36 disposed between the common iliac veins 10 at the lower end of the inferior vena cava 12. In contrast, the enlarged inboard anchor crowns 40″ of the embodiments shown in FIGS. 5 and 6 are disposed distally of the proximal end of the stent 38, hence being located securely within the consistently circular or elliptical cross-section of a common iliac vein 10 as shown in FIG. 7.

FIGS. 5 and 6 differ in the length of the anchor crowns 40″ in a direction parallel to the central longitudinal axis 44. Specifically, the length of all of the crowns 40 along the length of the stent 38 is uniform in FIG. 5. Conversely, FIG. 6 shows that at least one, in this example both, of the anchor crowns 40″ is of different length to, in this example longer than, the other, body crowns 40′of the stent 38.

The length of a crown 40, hence the length of struts 46 making up a crown 40, can be varied to match the radial force applied by struts 46 of adjacent crowns 40 within a sizing range. For example, struts 46 can be lengthened to reduce radial force or can be shortened to increase radial force locally, expressed as radially outward pressure per unit area.

FIGS. 8 and 9 illustrate an apparatus and a method for forming a stent 38 with a radially-expanded pair of anchor crowns 40″ as shown in FIGS. 5 and 6. The stent 38 is formed on an elongate cylindrical mandrel 62 as shown in FIG. 8 that fits within the lumen of the stent 38 as shown in FIG. 9. The forming step may conveniently be performed together with a final heat treatment step.

The mandrel 62 has an integral collar defining a shallow circumferential flange or band 64 defining a focal region of increased external radius. As can be seen in FIG. 8, the length of the band 64 corresponds to the combined length of a pair of anchor crowns 40″ of the stent 38 joined by short connectors 52 that bridge the gap 42 between them. The band 64 terminates at its ends in radiused or chamfered steps or shoulders 66 that align longitudinally with the gaps 42 between the anchor crowns 40″ of the pair and the adjacent body crowns 40′of the stent 38 immediately outboard of the pair. Specifically, the shoulders 66 align with the chicanes 56 of the long connectors 50 that bridge those gaps 42.

Thus, when the stent 38 is pressed radially inwardly against the mandrel 62 and thereby plastically deformed to conform to the external shape of the mandrel 62, the radial protrusion of the band 64 generates the radial enlargement of the inboard pair of anchor crowns 40″ relative to the outboard body crowns 40′that is seen in FIGS. 5 and 6. The stent 38 retains this set shape upon being removed from the mandrel 62.

Longitudinal grooves 68 in the band 64 extend from a closed inboard end to an open outboard end where they intersect and cross the shoulders 66 of the band 64. The radius of the mandrel 62 at the base of each groove 68 corresponds to the radius of the mandrel 62 outboard of the band 64.

The grooves 68 in the band 64 are grouped in oppositely-facing pairs, angularly spaced around the circumference of the band 64 to match the angular positions of the long connectors 50 that extend trough-to-trough to join body crowns 40′to respective anchor crowns 40″ of the enlarged pair.

The grooves 68 of each pair approach each other at their inboard ends, but do not join, and in this example are offset angularly from each other to match the aforementioned angular offset between successive long connectors 50 along the length of the stent 38. The grooves 68 thereby accommodate the long connectors 50 to allow the long connectors 50 to bend smoothly outwards in the inboard direction without excessive stress as they bridge the gap 42 between the body crowns 40′and the radially-enlarged anchor crowns 40″. The step-like chicanes 56 of the long connectors 50 also help the long connectors 50 to effect a low-stress transition between the body crowns 40′and the radially-enlarged anchor crowns 40″.

As noted above, the shoulders 66 define locating formations 60 that effect a sharp, stepwise transition between the body crowns 40′and the radially-enlarged anchor crowns 40″. The formations 60 will strongly resist migration of the stent 38 in use. Advantageously, however, that sharp transition is effected without a correspondingly sharp transition in the long connectors 50, hence mitigating the challenges of stress and fatigue.

Moving on now to FIG. 10, the stent 38 in this embodiment shows that more than one pair of crowns 40 may be radially enlarged to serve as anchor crowns 40″, and that those crowns 40″ can be radially enlarged to different extents. In this example, the pairs of anchor crowns 40″ decrease in diameter in stepwise fashion in opposed outboard directions from a central pair of anchor crowns 40″ of maximum diameter. Thus, the stent 38 is convex if viewed in longitudinal section or side view. Again, in use of the stent 38, this configuration beneficially focuses maximum restraining pressure at locations inboard of ends of the stent 38. However, the longitudinal progression of diameter from one pair of crowns 40 to the next could be different to that shown in FIG. 10, for example with a concave profile increasing to two or more maxima in diameter spaced apart longitudinally along the stent 38.

Thus, FIG. 10 shows that the diameter of a stent 38 can vary along the length of the stent 38. This focuses outward radial force against the surrounding vessel wall as in the preceding embodiments but also enables the stent 38 to be tailored to suit not just the size but additionally, to some extent, the shape or contour of a target vessel around the lesion to be treated. This tailored fit and the resulting mechanical engagement provides additional anchoring of the stent 38 even if the stent 38 is sized incorrectly for the vessel.

This variation in diameter can be effected in a stepwise manner, as illustrated in FIG. 10, or in a more continuous manner without steps, for example if at least some of the crowns 40 have frusto-conical profiles like those shown in FIGS. 12 and 13. Again, as in FIG. 6, the length of struts 46 and hence of the crowns 40 could be modified from one crown 40 to the next to adjust radial force or pressure along the length of the stent 38.

The stepped shape of the stent 38 shown in FIG. 10 can be formed by a variant of the mandrel 62 shown in FIGS. 8 and 9, with a corresponding series of bands 64 that increase in diameter in a stepwise manner in opposed inboard directions. Such a mandrel 62 is shown in FIG. 11. The stepped outboard edges of the bands 64 are intersected by longitudinal grooves 68 in angular alignment with the long connectors 50 of the stent 38.

FIGS. 12 and 13 show stent variants 38 in which anchor crowns 40″ of an inboard pair connected to each other by short connectors 52 are flared in opposite outboard directions. In these variants, the orientation of some struts 46 is changed so that the associated anchor crowns 40″ flare away from the longitudinal centreline of the stent 38. In other words, each anchor crown 40″ of that inboard pair is plastically deformed such that its radially outermost circumferential face 58 transforms from a straight-walled cylindrical configuration of uniform diameter into a frusto-conical configuration whose diameter increases in the outboard direction. The outboard ends of the struts 46 of those anchor crowns 40″ are therefore lifted to protrude radially outwardly from the straight-walled cylindrical profile of the narrower body crowns 40′outboard of them.

The outboard apices 48 or peaks of the waveforms of the flared anchor crowns 40″ thereby create teeth or barbs of oppositely-facing, divergent serrated edges 70. Those edges 70 have a similar locating function to the flared ends 30 of the prior art stent 22 shown in FIG. 3, but the inboard location of the edges 70 is beneficial for the same reason as discussed in relation to the enlarged inboard anchor crowns 40″ of FIG. 7.

In FIG. 12, the flared anchor crowns 40″ are also radially enlarged in the manner of the arrangements shown in FIGS. 5 and 6, whereas in FIG. 13 the anchor crowns 40″ are flared without radial enlargement other than due to the flaring itself. Thus, in FIG. 12, the longitudinally inboard edges of the flared anchor crowns 40″ lie on a radius outside that of the outboard body crowns 40′but in FIG. 13, the longitudinally inboard edges of the flared anchor crowns 40″ lie on the same radius as the outboard body crowns 40′. In other words, there is no change in diameter of the stent 38 from one crown 40 to the next but only a change in orientation of at least some struts 46 relative to lines parallel to the longitudinal central axis 44 of the stent 38.

When a stent 38 of FIG. 12 or 13 is in situ within a common iliac vein 10 or other vessel as represented in FIG. 14, the serrated edges 70 of the flared crowns 40 dig into and engage mechanically with the surrounding wall of the vein 10. Once its edge 70 is engaged with the wall of the vein 10, the leading or downstream flared anchor crown 40″ relative to the direction of blood flow will tend to widen or splay to resist downstream movement of the stent 38. Additional mechanical engagement between the stent 38 and the vein 10 is provided by the stepped, radially-enlarged profile of the flared anchor crowns 40″ shown in FIG. 12. Those anchor crowns 40″ protrude radially from the surrounding body of the stent 38 to emboss the inner wall of the vein 10 with recesses 72 that complement and receive the protruding parts of the anchor crowns 40″.

It would be possible for more than one pair of anchor crowns 40″ to be flared in the manner shown in FIG. 12 or 13, or for the diameters or lengths of those flared anchor crowns 40″ to vary along the length of the stent 38 like those shown in FIG. 5, 6 or 10, or for flared anchor crowns 40″ to be combined with other radially-expanded but non-flared anchor crowns 40″ in the same stent 38. It would also be possible for two or more of the anchor crowns 40″ to have matching frusto-conical inclination so that the resulting stent 38 could have unidirectional location properties, for example to resist displacement preferentially in a downstream direction when oriented in a vessel such that the flares of those anchor crowns 40″ open outwardly in that direction.

Again, the flared shapes of the anchor crowns 40″ shown in FIGS. 12 and 13 can be imparted to the stent 38 by a mandrel 62 like that shown in FIGS. 8 and 9 but with the band 64 of the mandrel 62 shaped to produce and hence to complement the desired shape of the anchor crowns 40″. In this instance, as shown in FIG. 15, a circumferential band 64 of the mandrel 62 has a waisted shape defined by a shallow circumferential groove 74 defining a concave formation between frusto-conical faces 76 that taper in the inboard direction toward their mutual interface. FIG. 16 shows how the anchor crowns 40″ aligned with the band 64 may be pressed into the groove 74, in this case by tension applied to a circumferential loop 78 of wire encircling the stent 38 and the mandrel 62 around the waist of the band 64. This reduces the diameter of the loop 78 around the stent 38, particularly in alignment with where short connectors 52 join the anchor crowns 40″ that are to be flared oppositely to each other.

It will be apparent that the flared but not otherwise radially expanded variant of the stent 38 shown in FIG. 13 can also be made by a mandrel 62 similar to that shown in FIGS. 15 and 16. In that case, however, the base of the groove 74 would be on the same radius as the portions of the mandrel 62 outboard of the band 64.

There could be two or more bands 64 spaced longitudinally along the mandrel 62. It would also be possible for one or more of the bands 64 to have only one frusto-conical face 76. In this way, one anchor crown 40″ could be flared and other crowns 40 of a stent 38 could be of constant diameter along their length. For example, one anchor crown 40″ of an inboard pair of crowns 40 could be flared and another crown 40 of the pair could be of constant diameter, whether of the same diameter as body crowns 40′ outboard of the pair or stepped to be an anchor crown 40″ of greater diameter than the body crowns 40′. It would also be possible for one frusto-conical face 76 of the mandrel 62 to underlie two or more anchor crowns 40″ of the stent 38 so that both of those anchor crowns 40″ are correspondingly flared in succession, potentially with their flared faces in mutual angular alignment.

FIG. 17 shows a sixth embodiment of the invention to illustrate that more than one portion may be enlarged radially and/or longitudinally along the length of the stent 38. In this example, enlarged portions 40″ are spaced apart longitudinally along the stent 38 and separated by body portions 40′that are not similarly enlarged.

Turning finally to FIGS. 18a and 18b, these drawings show how a balloon-expandable stent 38 can be expanded radially into a stepped profile shown in FIG. 18b. This is achieved by inflation of a correspondingly-stepped balloon 80 disposed in the lumen of the stent 38 as shown in FIG. 18a.

When the stent 38 has been conveyed to a target location in a blood vessel 10 as shown in FIG. 18a, inflation and radial expansion of the balloon 80 also expands a radially-enlarged circumferential band 82 of the balloon 80. For example, the wall thickness of the balloon 80 may be increased locally to define the band 82. The balloon 80 and the band 82 then bear outwardly against the interior of the stent 38 to impart a correspondingly stepped profile in the stent 38 that engages the wall of the vessel 10 as shown in FIG. 18b, in a manner analogous to that shown in FIG. 14.

FIGS. 17, 18a and 18b exemplify that the stent need not have a segmented configuration and could, for example, comprise a continuous tubular wall that could be woven or formed of a mesh.

Many other variations are possible within the inventive concept. For example, the struts of a crown may follow a differently oscillating waveform such as an undulating waveform exemplified by a sine wave. More generally, the transitions or apices where the struts join in circumferential succession could be rounded or radiused rather than sharp or acutely angled as shown in FIGS. 5, 6, 10, 12 and 13. Also, the struts need not be straight as shown in FIGS. 5, 6, 10, 12 and 13 but could be curved either continuously along their length or with one or more smooth or sharp inflections or bends along their length, hence being wavy, kinked, dog-legged or chicaned.

The number of connectors could vary along the length of the stent. For example, additional connectors could be used between crowns at or near to the ends of the stent to add stiffness to the skeletal frame structure. Indeed, additional connectors could be used anywhere else where added stiffness may be beneficial, such as between the anchor crowns of a radially-expanded and/or flared pair or other group.

Whilst it is preferred that radially-expanded and/or flared anchor crowns are grouped in pairs joined by short connectors, there could be more than two of such anchor crowns in a group joined by short connectors if the outermost anchor crowns of the group are joined by long connectors to body crowns immediately outboard of the group.

Conversely, a single radially-expanded and/or flared anchor crown could be joined by long connectors to one adjacent body crown and by long connectors or short connectors to another adjacent crown. In this respect, short connectors may be appropriate at the radially inward end of a flared anchor crown, particularly if that anchor crown is not otherwise expanded radially as in FIG. 12.

In a broad sense, the invention is not limited to segmented stents and so can encompass a stent with an enlarged portion that does not correspond to one or more segment units. For example, the invention could be applied to a woven stent as noted above, or to a mesh-patterned stent or a spiral crown stent.

1. A stent comprising a tubular body longitudinally extending between a proximal open end and a distal open end, wherein:
the tubular body has an enlarged portion extending longitudinally between the proximal open end and the distal open end and has a radially enlarged configuration relative to at least both longitudinally adjacent portions in the tubular body in an expanded state.

2. The stent of claim 1, wherein the enlarged portion is pre-formed with a radially enlarged configuration relative to the proximal open end and the distal open end.

3. The stent of claim 1 or claim 2, comprising a longitudinal series of circumferential tubular segments that are separated by, and alternate with, gaps along the length of the stent between opposed end segments of the series, wherein:
each segment of the series comprises struts that are disposed in a circumferentially extending waveform arrangement;
successive segments of the series are interconnected by connectors that bridge the respective gaps; and
segments of the series disposed between the end segments comprise at least one anchor segment that has in an expanded state a radially enlarged configuration relative to at least one interconnected adjacent body segment.

4. The stent of claim 3, wherein at least one anchor segment is of substantially constant diameter along its length in a longitudinal direction.

5. The stent of claim 3 or claim 4, wherein at least one anchor segment is flared along its length in a longitudinal direction.

6. The stent of claim 5, wherein the or each flared anchor segment expands in a longitudinal direction toward one of the end segments.

7. The stent of claim 5 or claim 6, comprising at least two anchor segments that are flared in opposed longitudinal directions.

8. The stent of any of claims 3 to 7, wherein the connectors extend between respective apices of the waveform arrangements of successive segments.

9. The stent of claim 8, wherein trough-to-trough connectors extend between facing troughs of the waveform arrangements of successive segments.

10. The stent of claim 9, wherein the trough-to-trough connectors connect at least one anchor segment to at least one body segment of the series.

11. The stent of any of claims 8 to 10, wherein peak-to-peak connectors extend between facing peaks of the waveform arrangements of successive segments.

12. The stent of claim 11, wherein the peak-to-peak connectors connect successive anchor segments and/or successive body segments of the series.

13. The stent of any of claims 3 to 12, wherein the connectors have opposed bends or inflections in succession along their length.

14. The stent of any of claims 3 to 13, wherein at least two of the anchor segments are grouped in longitudinal succession.

15. The stent of any of claims 3 to 14, wherein at least one anchor segment extends longitudinally to a substantially different extent than at least one other segment of the series.

16. The stent of any of claims 3 to 15, comprising a group of anchor segments whose radius increases and/or decreases along the group.

17. The stent of any of claims 3 to 16, comprising at least two anchor segments that are spaced apart longitudinally along the series and separated by least one body segment.

18. The stent of any of claims 3 to 17, wherein the or each body segment is of substantially the same diameter as the end segments.

19. The stent of any preceding claim, wherein a stepped outer profile is defined between the enlarged portion and the longitudinally adjacent portions.

20. The stent of claim 19, wherein the stepped outer profile comprises a radially-extending circumferential shoulder.

21. The stent of claim 19 or claim 20, wherein the stepped outer profile is circumferentially serrated.

22. The stent of claim 21, wherein serrations of the stepped outer profile are defined by a waveform arrangement of struts.

23. A method of manufacturing a stent, the method comprising:
inserting a mandrel into a lumen of the stent, the mandrel comprising at least one radially-protruding circumferential band;
aligning at least one portion of the stent longitudinally with the or each band of the mandrel, the or each of those aligned portions of the stent being inboard of opposed end portions of the stent; and
by effecting relative radial movement between the stent and the mandrel, forming the stent around the mandrel such that the or each portion aligned longitudinally with the or each band is enlarged radially relative to other portions that are not aligned longitudinally with the or each band.

24. The method of claim 23, wherein the portions of the stent comprise a longitudinal series of interconnected circumferential tubular segments and the method comprises;
aligning at least one of the segments of the stent longitudinally with the or each band of the mandrel, the or each of those aligned segments being inboard of opposed end segments of the stent; and
by effecting relative radial movement between the segments and the mandrel, forming the segments around the mandrel such that the or each segment aligned longitudinally with the or each band is enlarged radially relative to other segments that are not aligned longitudinally with the or each band.

25. The method of claim 24, comprising aligning at least one circumferential edge of the or each band with a respective gap between successive segments of the series.

26. The method of claim 25, comprising accommodating connectors extending between the successive segments in respective angularly-spaced grooves of the band intersecting the or each circumferential edge.

27. The method of any of claims 24 to 26, wherein each segment of the series comprises struts that are disposed in a circumferentially-extending waveform arrangement and interconnection between a segment aligned with the band and an adjacent segment not aligned with the band is effected by connectors that extend between facing troughs of their waveform arrangements.

28. The method of claim 27, wherein interconnection between segments of a group of segments all aligned with one band is effected by connectors that extend between facing peaks of their waveform arrangements.

29. The method of any of claims 22 to 28, comprising longitudinally aligning a group of two or more successive segments of the series with one band that is aligned with all segments of the group.

30. The method of claim 29, comprising pressing the segments of the group into conformity with a concave formation of the band by applying radially inward pressure locally to connectors extending between those segments.

31. The method of any of claims 21 to 30, comprising forming a stepped profile between a portion of the stent that is aligned with the, or one of the, bands and an interconnected or adjoining portion of the stent that is not aligned with that band.

32. The method of any of claims 21 to 31, comprising forming a flared profile in the or each portion of the stent that is aligned with the or each band.

33. The method of claim 32, comprising forming a step between a portion of the stent with a flared profile and an interconnected or adjoining portion of the stent.
